(19) United States
(12) Patent Application Publication
(10) Pub. No.: US 2009/0101730 A1
(43) Pub. Date: Apr. 23, 2009

(54) Vented Dispensing Bottle/Cap Assembly

(76) Inventors: Brian T. Davis, Burlington, WI (US); Ranjit A. De Silva, Racine, WI (US); Jamie T. Huynh, Franklin, WI (US); William G. Parsons, Racine, WI (US); Clint J. Rodriguez, Antioch, IL (US); Murthy S. Munagavalasa, Racine, WI (US)

Correspondence Address: S.C. JOHNSON & SON, INC., 1525 HOWE STREET, RACINE, WI, 53403-2236, US

(21) Appl. No.: 12/033,181

(22) Filed: Feb. 19, 2008

Related U.S. Application Data
(60) Provisional application No. 60/981,171, filed on Oct. 19, 2007.

(51) Int. Cl. A61L 9/12

(52) U.S. Cl. 239/44

(57) ABSTRACT

A refill is disclosed for an electronically activated dispenser of liquid volatiles, such as insecticides and fragrances. There is a bottle having a wick, a wick holder, a vent hole, an outlet, and a removable cap. When the cap is in place it covers both the wick and a vent hole. The cap seals the vent hole via an interference fit with a well adjacent the vent hole and also either a direct abutment against the vent hole or a surrounding of the vent hole. Also disclosed is a refill where the porous wick and vent hole are within defined size ranges so as to facilitate the use of a pyrethrum insecticide.

### CROSS-REFERENCE TO RELATED APPLICATIONS

This application claims priority based on U.S. provisional application 60/981,171 filed on Oct. 19, 2007.

### STATEMENT REGARDING FEDERALLY SPONSORED RESEARCH/DEVELOPMENT

Not applicable.

### BACKGROUND OF THE INVENTION

The present invention relates to refill bottles for air treatment dispensers. More specifically, it relates to particular cap and venting structures incorporated into such refill bottle assemblies.

A wide variety of volatile air treatment chemicals (e.g. insect control agents such as insecticides or insect repellents; fragrances; deodorizers; etc.) are dispensed from electrically heated dispensers. A bottle or other container of the air treatment chemical has a wick extending into it that draws the air treatment chemical out of the bottle/container to a wick end positioned next to a heater. The heat from the heater then causes the air treatment chemical to volatize into the air.

Examples of such dispensers are disclosed in U.S. Pat. Nos. 4,663,315, 5,038,394, 5,095,647, 5,222,186, 5,290,546, and 5,647,053. See also EP1,825,748.

An especially desirable form of such dispensers inserts a replaceable bottle and wick assembly into the bottom of a heater arrangement. See e.g. U.S. Pat. No. 6,968,12.4. Once the air treatment chemical is used up, the bottle and wick subassembly can be detached from the heater and replaced with a refill. Hence, the heater, which has a much longer useful life, need not be disposed of when the active in one bottle is used up.

For each intended use, the appropriate/optimized chemical ingredients are selected to form a volatile liquid. Typically the active is dissolved in a volatile solvent carrier as well. A particularly desirable insect control active is pyrethrum extract. A pyrethrum solution is drawn up a porous wick extending into the bottle (via capillary action) to a portion of the wick outside of the enclosed bottle. A plug-in type heating element increases the temperature of a zone around the outer portion of the wick, volatizing the pyrethrum.

However, if one uses a typical fine-pored wick with pyrethrum, pyrethrum will tend to clog the wick prior to the active being used up. By using wicks with larger pores (see generally EP 1,825,748) the clogging can be minimized or avoided. However, that can cause other problems. For example, if the bottle is sealed and is subjected to heat, elevation changes, or other common circumstances that increase the pressure of the contents, liquid from the reservoir can be put under pressure such that the liquid is forced up through the wick to pool on top of the bottle causing waste and/or drooling or pooling problems. If one attempts to vent the bottle to avoid this effect, using a standard hole, this can create a leakage site during storage or shipment. Independent of that issue, if such a hole is too small, liquid in the vent hole can have a surface tension sufficient to form a liquid plug, tending to block the vent hole.

In U.S. Pat. No. 6,446,880 (see also the related U.S. Pat. No. 6,386,462) there was described a piezoelectric vibrator linked to a reservoir for a volatile by a tapering wick. The reservoir was provided with a wick holder that had a vent hole, and there was also a closure cap to seal off the reservoir from the air prior to use. However, that vent hole, even when covered by the cap, did not prevent the bottle contents from bypassing the wick and prematurely reaching the upper structure in greater than optimal quantity. Further, the wick structure tapered so greatly in this design as to make it more difficult to use with certain actives that were not fragrances.

In U.S. Pat. No. 6,786,427 there was described another reservoir for a volatile in which another wick holder had a vent hole. Further, there was shown a closure cap that sealed off the reservoir from the air by insertion of a cap portion into a well formed in the wick holder. However, here the seal formed by the cap and the well depended solely on an interference fit between certain cap structures and the well, requiring precise part sizes to achieve a secure seal and presenting only one means for sealing the vent hole without any back-up sealing structure should the first prove inadequate. Furthermore, the structures were not optimal for use with pyrethrum.

More complex solutions to the venting issue may raise the cost of the refill unacceptably.

Thus, there is a need for improved refill/wick assemblies for such dispensers, particularly when pyrethrum is an active in the bottle.

### BRIEF SUMMARY OF THE INVENTION

The present invention provides a refill for an electrically activated dispenser of liquid volatiles, such as insecticides and fragrances, with an improved sealable venting system. In one aspect there is a refill for an electrically activated dispenser of liquid volatile. The refill is of the type that has a bottle having an outer housing wall, a vent hole, and an internal cavity, the outer housing wall having a main upper outlet. There are also a porous wick mounted relative to the bottle so as to have one end extending into the internal cavity and another end extending outside the bottle, and a cap linked to the bottle so as to have a portion of the cap removably cover the vent hole.

In one form of the invention the improvement is that the porous wick has a plurality of pores, at least some of the pores having a diameter of between 15 microns and 45 microns, the vent hole is between 0.2 mm and 0.5 mm (e.g. 0.2 mm-0.4 mm) in diameter, and a liquid volatile that includes pyrethrum (e.g. 2 to 8 percent by weight of pyrethrum in a hydrocarbon solvent) is positioned in the internal cavity.

In preferred forms there is a wick holder portion of the bottle that extends across the outlet and that has the vent hole through it. The vent hole is positioned at a lower end of a well portion of the wick holder portion, and the cap has a depending band that is suitable to seal the vent hole by contacting both side walls and a bottom wall of the well portion. For example, with the vent hole located in the bottom wall of the well portion, the band can have a cupped lower contact surface sized to span the vent hole and thus provide a sealing contact with the bottom wall on either side of the vent hole, and the cap can cover both an upper end of the wick and the vent hole and thereby prevent liquid in the reservoir from passing through the vent hole either directly to an upper portion of the wick or to the outside of the bottle, beyond the cap.

In another aspect the invention provides a different form of refill for an electrically activated dispenser of liquid volatile. In this form the refill is of the type that has a bottle having an outer housing wall, a vent hole, and an internal cavity, the outer housing wall having a main upper outlet. Such refills have a porous wick mounted relative to the bottle so as to have one end extending into the internal cavity and another end extending outside the bottle, and a cap linked to the bottle so as to have a portion of the cap removably seal the vent hole and the end of the wick extending outside the bottle.

In this aspect of the invention the improvement relates to a wick holder portion of the bottle that extends across the outlet and supports the porous wick, the wick holder portion having the vent hole through it, wherein the vent hole is positioned at a lower end of a well portion of the wick holder portion and the cap has a depending band that is suitable to seal the vent hole by contacting both the well side walls and a bottom wall of the well.

The refills of the present invention provide an assembly that can be stored and shipped without leakage or evaporation concerns (as the cap covers both the wick end and the vent hole). Removal of the cap exposes both the vent hole and the wick end for use.

Importantly, this is achieved in an inexpensive manner that permits pyrethrum to be used with large pore size wicks with reduced adverse side effects. For example, clogging issues are essentially avoided without incurring drooling or pooling issues caused by spikes in pressure within the refill bottle.

The foregoing and other advantages of the present invention will be apparent from the following description. In that description reference is made to the accompanying drawings, which form a part thereof and in which there is shown by way of illustration, and not limitation, preferred embodiments of the invention. Such embodiments do not necessarily represent the full scope of the invention.

### BRIEF DESCRIPTION OF THE DRAWINGS

FIG. 1 is a front, right, upper perspective view of a refill assembly in accordance with the present invention;

FIG. 2 is a view similar to FIG. 1, but with a cap portion exploded upward there from;

FIG. 3 is a vertical cross-sectional view of the FIG. 1 assembly;

FIG. 4 is a view similar to FIG. 3, but with a cap exploded there from;

FIG. 5 is a vertical cross-sectional view of the FIG. 4 bottle (without cap) installed in a plug-in type electrical heater;

FIG. 6 is an enlarged sectional view of the region highlighted with arrows in FIG. 3; and

FIG. 7 is a view similar to FIG. 6, except of a second embodiment identical to the first except for a depending band having a cupped contact surface.

### DETAILED DESCRIPTION OF THE INVENTION

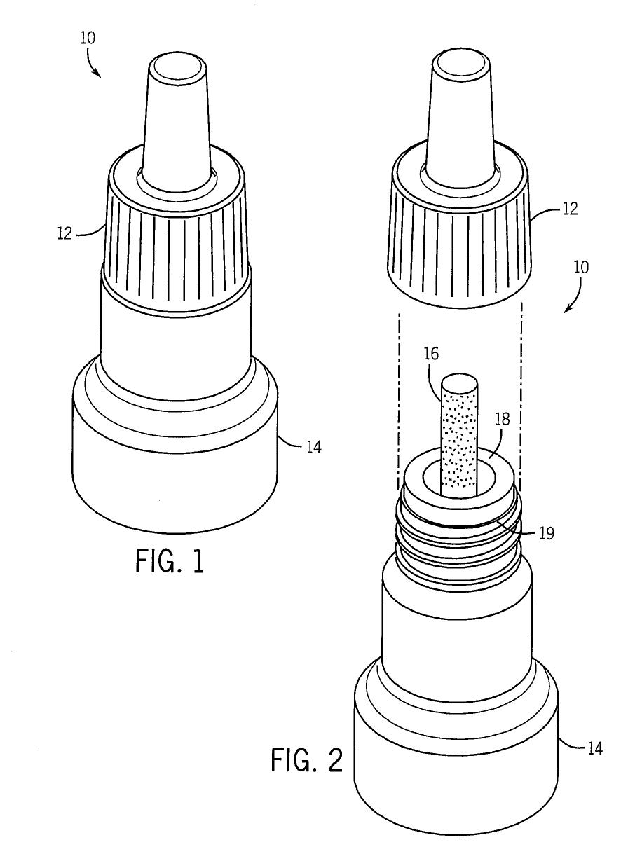

Referring first to FIGS. 1-4, a refill assembly, shown generally at 10, according to the present invention is shown that has a cap 12 and a bottle 14. In FIG. 1 the cap 12 is fastened on the top of the bottle 14 to secure the volatile liquid inside the bottle 14. In FIG. 2, the cap 12 is shown as having been removed from bottle 14 to reveal a porous wick 16 that is held in place by a wick holder 18 that spans a bottle outlet 19.

A preferred volatile chemical agent for use therewith is an insecticide solution containing 2%-8% natural pyrethrum. For example, one can formulate a mosquito control formulation that has 94.36% isoparaffinic hydrocarbon (solvent), 2.31% pyrethrum (insecticide), 2.00% 2,6 di-t-butyl-p-cresol (an antioxidant a/k/a BHT), 1.29% prallethrin (another insecticide), and 0.04% d-limonene (a fragrance). It is contemplated that the volatile liquid may be composed of other chemical agents that provide different functions.

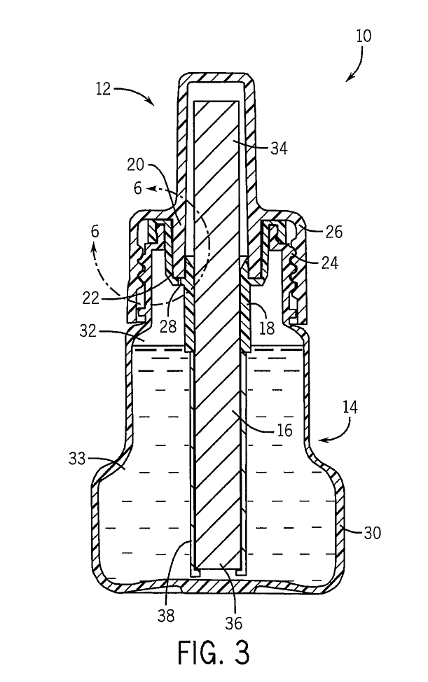

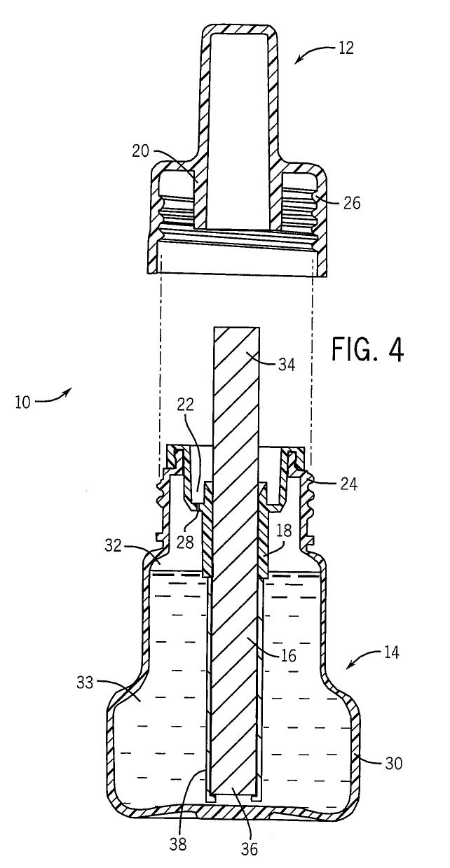

FIG. 3 shows cap 12 attached to the bottle 14 during storage and shipment to cover the outlet 19 and the portion of the porous wick 16 that extends outside of the bottle 14. As shown in FIG. 4, immediately prior to use the cap 12 is removed from the bottle 14 to expose the porous wick 16.

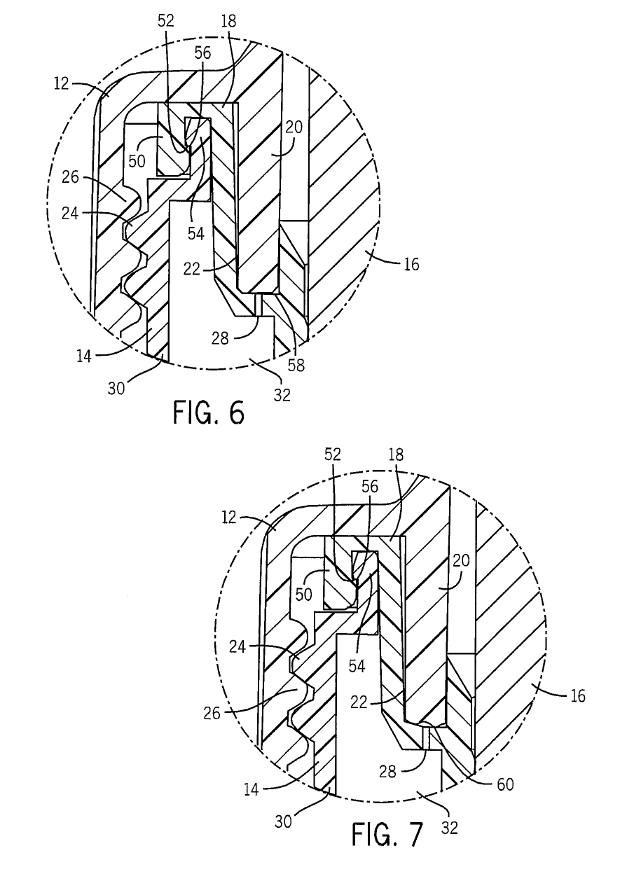

The cap 12 includes a depending band 20 that is inserted into a well 22 of the wick holder 18 when threads 24 of the cap 12 are sufficiently engaged into the threads 26 of the bottle 14 to cover the outlet 19. A vent hole 28 is located at the bottom of the well 22 of the wick holder 18, such that when the depending band 20 is sufficiently inserted into the well 22, the depending band seals the vent hole 28. The depending band 20 makes contact in or around the vent hole 28, and that portion either may be flat as shown in FIG. 6, or cupped as shown in FIG. 7.

The bottle 14 includes an outer wall 30 and an inner cavity 32 that contains volatile liquid 33. Note that if the cap 12 is attached to the bottle 14 as shown in FIG. 3, then the vent hole 28 is effectively sealed and the volatile liquid 33 and its associated vapors are contained within the inner cavity 32. However, if the cap 12 is removed from the bottle 14 as shown in FIG. 4, then the internal cavity of the bottle is vented through the vent hole 28 into the surrounding atmosphere if pressures in the bottle start to rise.

While in the illustrated embodiment vent hole 28 is shown as being located on a separate wick holder 18, it is contemplated that the vent hole 28 may be positioned in other locations. For example, the wick holder 18 may be integrated into the outer wall 30. Alternatively, even when the wick holder is separate, the vent may be in a wall of the bottle.

FIGS. 3 and 4 also illustrate a preferred configuration of a porous wick 16 that extends from an outer end 34 located outside of the bottle 14 to an inner end 36 located near the bottom of the inner cavity 32 of the bottle 14 so as to dip into the volatile liquid 33. The sides of the wick 16 inside inner cavity 32 may be covered by a vapor-impervious sleeve 38, albeit leaving the lower end in contact with the liquid. The sleeve 38 can be made of a reinforcing, but flexible material, such as polyethylene terephthalate (PET). This insures that the fragile porous wick 16 can be easily inserted into the sleeve 38, but also be protected from breakage as it is inserted into the outlet of the bottle.

The porous wick 16 may be formed of many conventional materials, such as porous ceramics, bonded fibers, and sintered plastics. We particularly prefer sand core or sintered glass wicks. We also prefer when using pyrethrum solutions that the porous wick 16 should have pore diameters in the range of 15 microns to 45 microns.

Linkage of the cap 12 and bottle 14 by their respective threads 24 and 26 are only one means of linking the cap 12 and bottle 14. Other linkage and fastening means may be used to link the cap 12 and bottle 14. For example, a bayonet fastening system may be used to link the cap 12 to the bottle 14.

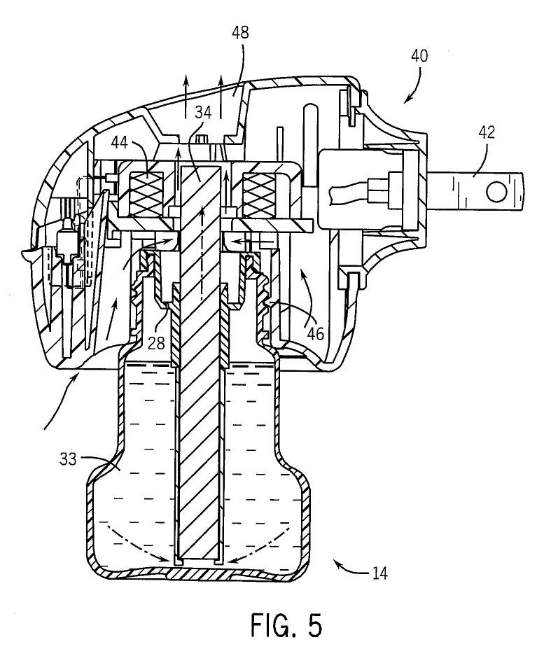

FIG. 5 depicts a conventional electrical heater capable of using the refills of the present invention. See generally U.S. Pat. No. 6,968,124, the disclosure of which is incorporated by reference, for details about this type of heater. We note that the bottle 14, with the cap 12 removed as shown in FIG. 4, is inserted into the electric volatile dispenser 40, which has a plug 42 insertable into an electric wall outlet (not shown) to provide electric current to heat heating elements 44. The heating elements 44 are positioned such that when the threads 24 of the bottle 14 are sufficiently engaged into the threads 46 of the electric volatile dispenser 40, the outer end 34 of the porous wick 16 is surrounded by the heating elements 44.

When the electric volatile dispenser 40 receives an electric current, the heating elements 44 generate heat in the area around the outer end 34 of the porous wick 16. This heat will increase the rate of volatilization of the volatile liquid 33 that has ascended the porous wick 16 to the outer end 34 near the heating elements 44. As the volatile liquid 33 volatilizes and the vapor ascends out of the outlet opening 48 of the electric volatile dispenser 40, the capillary action of the porous wick 16 will bring more of the volatile liquid 33 to the outer end 34 of the porous wick 16. Thus, the use of the heating elements 44 accelerates the volatilization of the volatile liquid 33 and increases the rate at which the volatile liquid 33 that ascends the porous wick 16.

When the bottle 14 is inserted into the electric volatile dispenser 40, vent hole 28 remains open and unobstructed. As liquid 33 is consumed, any drop in pressure within the bottle 14 that might otherwise result is relieved by air entering via the vent hole 28. Likewise, if pressure starts to build up in the cavity 32, the vapors can vent out vent hole 28, preventing unduly fast wicking.

FIGS. 6 and 7 show detailed cross-sectional views of the contact between the cap and wick holder adjacent the vent hole. Note that depending band 20 is inserted into the well 22 via the engagement of the threads 24 and 26. A radial lip 50 of the wick holder has a snapping tab 52 that interlocks with a radial lip 54 having a tab 56 on the outer wall 30. The radial lip 50 of the wick holder 18 is formed in an upside-down u-shape such that it can wrap up and over the radial lip 54 of the outer wall 30.

When the radial lip 50 of the wick holder 18 is pushed down over the radial lip 54 of the outer wall 30, the u-shape of radial lip 50 temporarily deforms until the snapping tabs 52 and 56 interlock and the radial lip 50 returns to a form substantially similar with its initial shape. In this manner, the wick holder 18 is connected to the outer wall 30 of the bottle 14.

FIGS. 6 and 7 also show in detail the depending band 20 inserted into the well 22 to seal the vent hole 28. In FIG. 6, the depending band 20 has a flat portion 58 on the bottom of the depending band 20 that is large enough to cover the entire vent hole 28 when the depending band is fully pressed into the well 22. Hence, sealing occurs both along the sides of the band and at its lower end.

Another configuration is shown in FIG. 7, in which a cupped surface 60 on the bottom of the depending band 20 substantially covers the vent hole 28 as the depending band 20 is pressed into the well 22. In the case where the cupped surface 60 substantially covers the vent hole 28, the cupped surface 60 will ideally promote predictable contact points between the depending band 20 and the well 22.

The preferred diameter for the vent hole 28 for a liquid volatile containing pyrethrum is between 0.2 mm and 0.5 mm. This range of diameters is selected because experimental data suggests that size to be sufficient to avoid blockage by a liquid droplet while also being sufficient to provide pressure balance within the bottle 14 when in use within a volatile dispenser 40.

The depending band 20 and the well 22 preferably have dimensions and tolerances such that when the depending band 20 is inserted into the well it has a tight interference fit against the lower sidewalls of the well. This arrangement provides an additional barrier to loss of liquid through the vent hole 28 when the cap 12 is in place on the bottle 14. The presence of such an interference fit makes advantageous the manufacture of the cap 12 and the wick holder 18 out of somewhat elastic material so as to reduce the amount of force require to assemble the cap to the bottle 14 or to remove it since a portion of the force will be required to rotatably push the depending band 20 down into the well 22 or to pull it back out.

While preferred embodiments of the present invention have been described, other embodiments of the invention are within the spirit and scope of this disclosure. Hence, the claims, when presented, should not be construed as being limited to just the disclosed preferred embodiments.

### INDUSTRIAL APPLICABILITY

The present invention provides a refill for an electronically activated dispenser of liquid volatiles, that has a vent hole associated with a refill bottle that is sealed during shipment and storage via a cap.

What is claimed is:

1. A refill for an electrically activated dispenser of liquid volatiles, the refill comprising:
a bottle having an outer housing wall, a vent hole, and an internal cavity containing the liquid volatile to be dispensed, the outer housing wall having a main upper outlet;
a porous wick mounted relative to the bottle so as to have one end extending into the internal cavity and another end extending outside the bottle; and
a cap linked to the bottle so as to have a portion of the cap removably cover the vent hole;
the porous wick having a plurality of pores, at least some of the pores having a diameter of between 15 microns and 45 microns; and
the vent hole being between 0.2 mm and 0.5 mm in diameter.

2. The refill of claim 1, wherein the liquid volatile comprises pyrethrum.

3. The refill of claim 1, wherein the liquid volatile comprises 2 to 8 percent by weight of pyrethrum and also comprises a hydrocarbon solvent.

4. The refill of claim 2, wherein the vent hole is between 0.2 mm and 0.4 mm in diameter.

5. The refill of claim 1, further comprising a wick holder portion of the bottle that extends across the outlet and wherein the vent hole is located in the wick holder portion.

6. The refill of claim 1, wherein the wick holder includes a well portion having side walls and a bottom wall, the vent hole is positioned at a lower end of the well portion, and the cap has a depending band that is suitable to seal the vent hole by contacting the side walls and the bottom wall of the well portion.

7. The refill of claim 6, wherein the vent hole is located in the bottom wall of the well portion and the band has a cupped lower contact surface sized to span the vent hole.

8. The refill of claim 1, in which the cap can cover both an upper end of the wick and the vent hole and thereby prevent liquid in the bottle from passing through the vent hole directly to an upper portion of the wick.

9. In a refill for an electrically activated dispenser of liquid volatile, the refill being of a type comprising:
a bottle having an outer housing wall, a vent hole, and an internal cavity, the outer housing wall having a main upper outlet;
a porous wick mounted relative to the bottle so as to have one end extending into the internal cavity and another end extending outside the bottle; and
a cap linked to the bottle so as to have a portion of the cap removably seal the vent hole and the end of the wick extending outside the bottle;
an improvement comprising:
a wick holder portion of the bottle that extends across the outlet and supports the porous wick, the wick holder portion having the vent hole through it, wherein the vent hole is positioned at a lower end of a well portion of the wick holder portion and the cap has a depending band that is suitable to seal the vent hole by contacting both well side walls and a bottom wall of the well.
